(19) United States
(12) Patent Application Publication
(10) Pub. No.: US 2017/0273623 A1
(43) Pub. Date: Sep. 28, 2017

(54) Adjustable and Detachable Cast with Pressure Sensor

(71) Applicant: KEEPSPRING MEDICAL INC, Taipei (TW)

(72) Inventors: Chung-Sheng Chen, Taipei (TW); Ching-Chuan Jiang, Taipei (TW); Yi-Jen Jiang, Taipei (TW)

(21) Appl. No.: 15/484,158

(22) Filed: Apr. 11, 2017

Related U.S. Application Data
(60) Provisional application No. 62/307,553, filed on Mar. 13, 2016.

(51) Int. Cl. A61B 5/00; A61B 5/03; A61B 5/026; A61F 5/01

(52) U.S. Cl. CPC A61B 5/6812; A61F 5/012; A61B 5/03; A61B 5/0022; A61B 5/746; A61B 5/026; A61B 2562/0247

(57) ABSTRACT

A cast equipment including a fixed element, a filling device with a first site fastened to the fixed element, and a pressure sensor attached to the filling device is disclosed in this application. The pressure sensor is able to detect and collect pressure data, and the fixed element has a first container and a shape-retaining moldable material inside the first container. The present application provides an equipment to detect a pressure data to a casted region. A method for using the cast equipment is also provided in this application.

### CROSS-REFERENCE TO RELATED APPLICATION

The present application claims the benefit of U.S. provisional Application No. 62/307,553 filed on Mar. 13, 2016, the contents of which are hereby incorporated by reference.

### FIELD OF THE INVENTION

The present application relates to a detachable cast, specifically an adjustable and detachable cast with pressure sensor to monitor a patient's bloods flows in casted body part. The present invention also relates to a method to ensure the patient's normal blood flow in casted body region by detecting a blockage of blood flow, and a secure and simple way to remove the cast when needed.

### BACKGROUND OF THE INVENTION

Bone fracture is a common medical condition that can occur to anyone at any stage of life. Stabilizing a fractured bone by means of a cast is an effective and low cost standard practice. However, the post fracture swelling makes cast too tight and creates pressure to casted body part. When swelling continues to occur after the cast has been placed and cause blood flow blockage, the cast often has to be split to relieve pressure as it may result in irreversible damage to the patient.

Removal of a conventional cast requires a saw blade, which is sharp and generates heat and particles in the air during sawing, poses risk and danger of cutting or burning the patient and inhalation of cast particles in the process.

One of the alternative removal methods is taught in U.S. Pat. No. 2,103,942, to Gillin. The Gillin is a detachable cast which applies zippers or hooks around the marginal edges and ensures ease of removal of cast. However the Gillin does not teach how to adjust the tightness of the cast if removal the cast is not an option.

On the other hand, a cast too tight can induce bad blood circulation and hence causes slower recovery of casted body part, while a cast too loose cannot stabilize a fractured bone and has no functionality. Nevertheless, swelling repeatedly occurs and abates after placing the cast so it can be hard to maintain normal blood flow while stabilizing fracture bone in the meantime during wearing a cast.

Additionally, swelling or blood flow blockage may occur without the awareness of the patient or doctor. A way to timely monitor so as to prevent irreversible damage to the patient is also needed.

It is accordingly the primary objective of the present invention that it provides an improved cast to adjust the pressure of the cast to the casted region. A further objective of the present invention is to provide an easier way to detach the cast when the need is detected.

### SUMMARY OF THE INVENTION

In one aspect, a cast equipment is provided in this application. The cast equipment includes a fixed element, a fixing device and a pressure sensor. The filling device with a first site is fastened to the fixed element, and the pressure sensor is attached to the filling device, and the pressure sensor is able to detect and collect pressure data.

According to the above, the fixed element has a first container and a shape-retaining moldable material inside the first container.

According to the above, the filling device has a second container and an inflatable balloon inside the second container.

According to the above, the first container and the second container are bags made of weave cloth.

According to the above, the filling device has a second site attached to the fixed element by weaving the first container and the second container together.

According to the above, the pressure sensor detects a pressure data through detecting the pressure of the filling device.

According to the above, the fixed element includes a separable fasten means and the separable fasten means is a zipper.

According to the above, the pressure sensor further includes a component for connecting to an internet environment, and the pressure sensor sends a collected pressure data to a user's device through the internet environment.

According to the above, the pressure sensor can send an alert to the user's device if the pressure sensor detects a pressure data over a threshold value.

According to the above, the threshold is 60 mmHg.

In another aspect, a method for detecting a pressure data to a casted region is also provided. The method has steps of (a) applying a fixed element having a separable fasten means to a patient; (b) fastening a filling device to said fixed element with an attached pressure sensor by the separable fasten means; and (c) detecting the pressure data by the pressure sensor.

According to the above, the method further has a step of sending a detected pressure data to a user's device.

According to the above, the detected pressure data is sent through a wireless network environment.

According to the above, the method further has a step of sending an alert to a user's device if the pressure data represents a blood blockage.

According to the above, the pressure data is a data over 60 mmHg.

In another aspect, a method for releasing a blockage to a casted region is provided. The method has steps of (a) applying a fixed element having a separable fasten means to a patient; (b) fastening a filling device to the fixed element with an attached pressure sensor by the separable fasten means; (c) detecting a blood blockage by the pressure sensor; and (d) deflating the filling device or removing the fixed element by the separable fasten means to release the blockage.

According to the above, the method further has a step of sending a detected blockage alert to a user's device after step (c).

According to the above, the detected blockage alert is sent through a wireless network environment.

According to the above, the filling device is an inflatable balloon.

According to the above, the blood blockage is a pressure data over 60 mmHg.

The spirit of the invention will be more readily appreciated by one of ordinary skilled in the art from the following drawings, working examples and descriptions.

### BRIEF DESCRIPTION OF THE DRAWINGS

FIG. 1 is a diagram of a cast equipment for one embodiment of the present application.

FIG. 2 is a diagram of a cast equipment on right leg with an inflating device attached to a filling device for a preferred embodiment of the present application.

FIG. 3(a) is a diagram showing a fixed element of the present application.

FIG. 3(b) is a diagram showing a filling device of the present application.

FIG. 4 is a diagram showing a fixed element attached to a filling device by weaving their containers together in the application.

FIG. 5 is a diagram of a cast equipment on torso with an inflating device attached to a filling device for a preferred embodiment of the present application.

FIG. 6 is a block diagram showing the steps of the method of detecting a pressure data and releasing a blood flow blockage to a casted region of the present application.

### DETAILED DESCRIPTION OF THE INVENTION

The features of the present application will become apparent from the following detailed description that is provided in connection with the accompanying figures of the invention. It should be understood that indicating embodiments of the present application are not intended to limit the scope of the following claims.

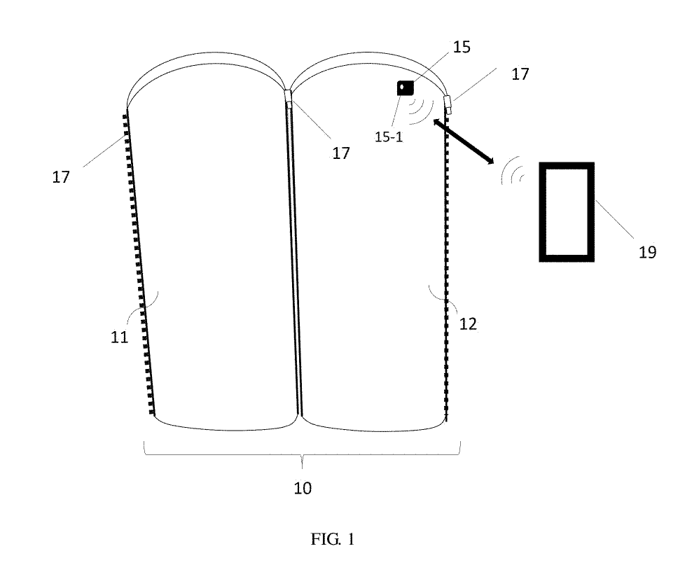

FIG. 1 shows a cast equipment 10 for one embodiment. The cast equipment 10 includes a fixed element 11 and a filling device 12. The fixed element 11 has a separable fasten means 17. The separable fasten means 17 in this embodiment is a zipper. One site of the filling device 12 is fasten to the fixed element 11 by the separable fasten means 17. There is a pressure sensor 15 attached to the filling device 12, and the pressure sensor 15 is able to detect and collect pressure data by detecting the pressure between the filling device 12 and a casted region of a patient. In pressure sensor 15 there is a component 15-1 which can connect to an internet environment and send a collected pressure data detected by pressure sensor 15 to a user's device 19 through internet environment.

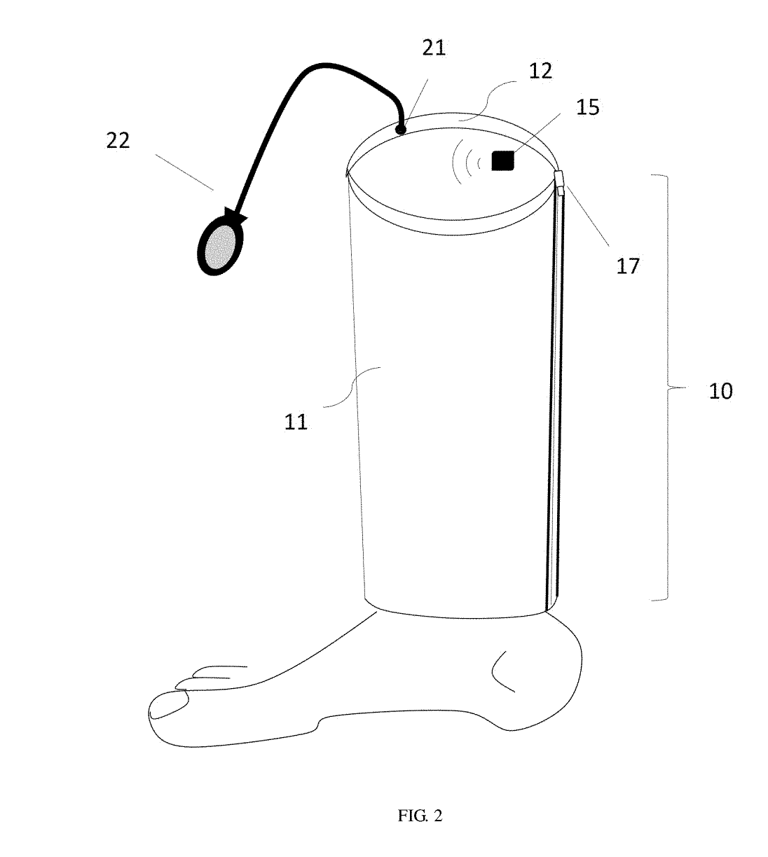

FIG. 2 shows a cast equipment 10 on right leg with an inflating device 22 connected to a filling device 12 for a preferred embodiment of the present application. Both sides of the fixed element 11 and the filling device 12 are fastened together by the separable fasten means 17 to make the right leg casted. If the pressure sensor 15 detects a pressure between the filling device and the casted leg is too high, or the patient himself feels the cast is too tight, the patient or doctor or others can deflate the filling device and release the pressure. The pressure may be caused by swelling of the bone fracture, once the swelling abates and hence makes the cast loosen, the inflating device 22 is able to inflate the filling device 12 to adjust again.

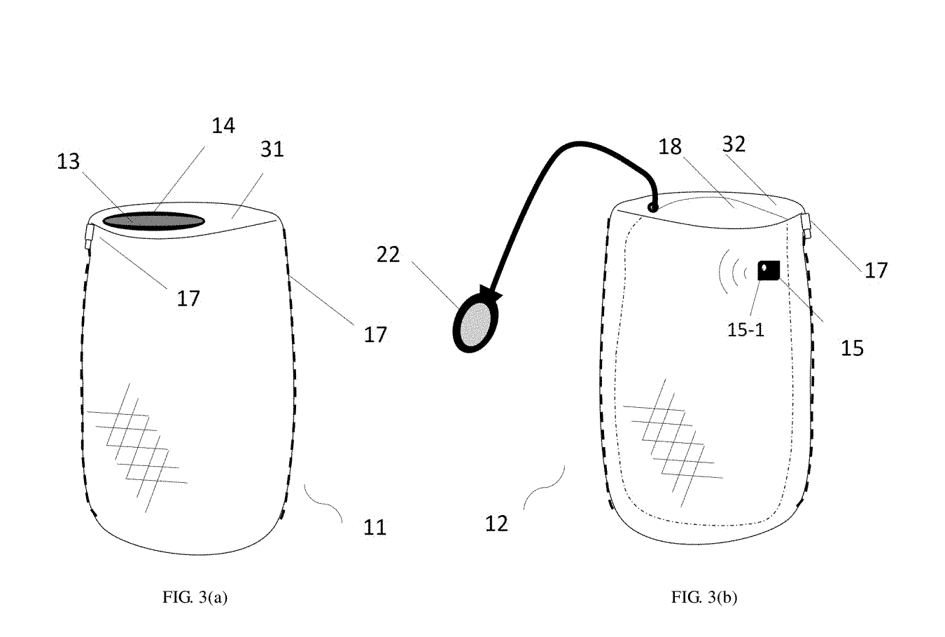

FIG. 3(a) shows an embodiment of a fixed element 11. The fixed element 11 has a first container 31 and a shape-retaining moldable material 13 injected into the fixed element 11 through the opening 14. The first container 31 can be a bag made of weaving cloth. The presenting application can fit the casted region and stabilize a fractured bone by the shape-retaining moldable material 13. In some embodiments, the shape-retaining moldable material 13 is plaster. There is a separable fasten means 17 set along the edge of the fixed element 11.

FIG. 3(b) shows an embodiment of a filling device 12. The filling device 12 has a second container 32 and an inflatable balloon 18 in the second container 32. The balloon 18 can be inflated or deflated by an inflating device 22. The second container 32 can be a bag made of weaving cloth. A pressure sensor 15 is attached to the filling device 12 and there is a component 15-1 in the pressure sensor 15 which can connect to an internet environment and send the pressure data collected by the pressure sensor 15. There is a separable fasten means 17 set along the edge of the filling device 12. The separated fixed element 11 and filling device 12 can be fastened together by the separable fasten means 17.

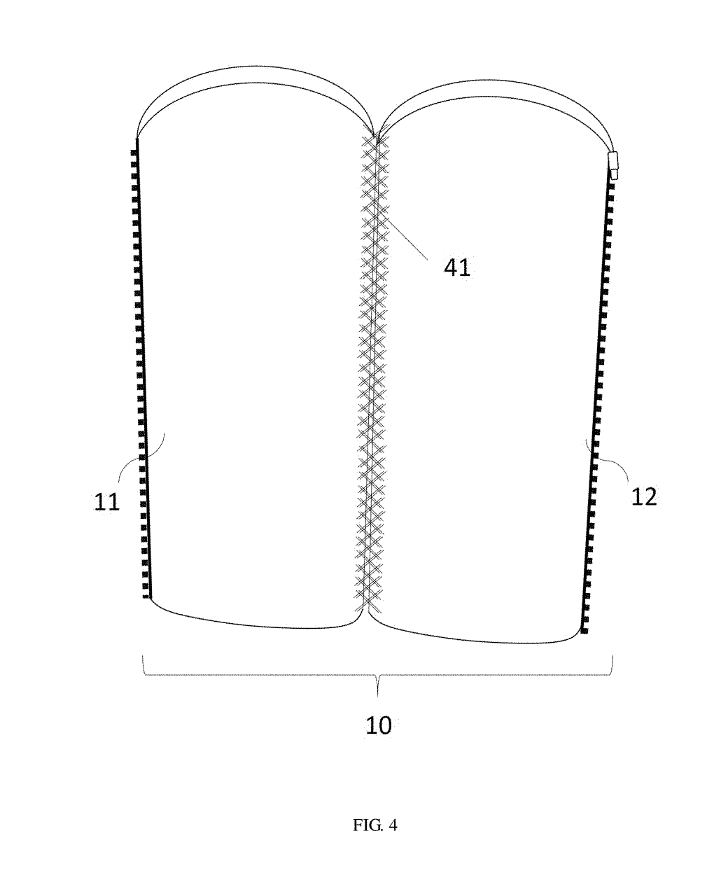

FIG. 4 shows that in some embodiment a fixed element is attached to a filling device by weaving their containers together with weaving thread 41.

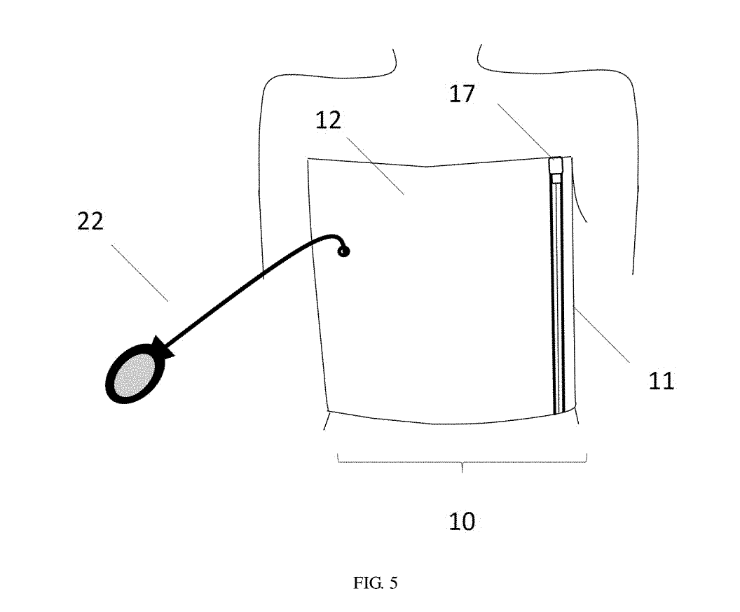

FIG. 5 shows a cast equipment 10 on torso with an inflating device 22 attached to a filling device 12 for a preferred embodiment of the present application. The inflating device 22 can inflate or deflate the filling device if the patient feels the cast is too loose or too tight. The size and shape of the fixed element 11 and the filling device 12 vary to fit the casted region. The fixed element 11 and the filling device 12 is attached by a separable fasten means 17.

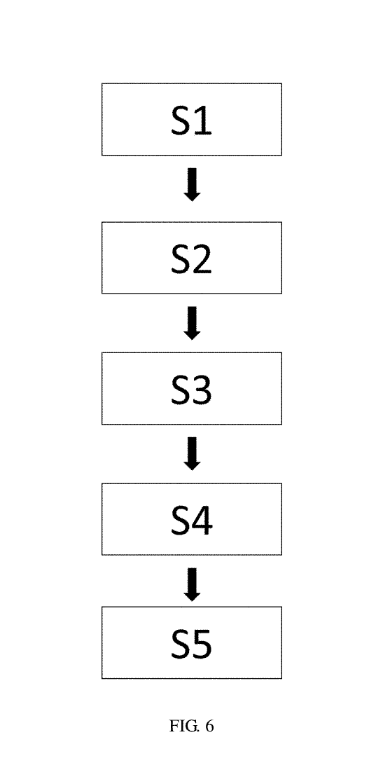

FIG. 6 is a block diagram showing the steps of the method of detecting a pressure data and releasing a blood flow blockage to a casted region of the present application. The method includes the steps of:

Step S1: Applying a fixed element having a separable fasten means to patient;

Step S2: Fastening a filling device to the fixed element with an attached pressure sensor by the separable fasten means;

Step S3: detecting a pressure data by the pressure sensor;

Step S4: sending the detected pressure data to a user's device through internet, or sending an alert to a user's device if the pressure data is over 60 mmHg, which is over a normal human diastolic pressure and is representing a blood flow blockage.

Step S5: Deflating the filling device or removing the fixed element by the separable fasten means to release a blockage.

The present invention applies a fixed element having a separable fasten means which are detachable and a filling device with pressure sensor which is able to release the tightness and the blood flow blockage caused by post-fracture swelling. The present application provides a way to adjust the cast and solve the problem in the field.

1. A cast equipment comprising:
a fixed element;
a filling device with a first site fastened to said fixed element, and
a pressure sensor attached to said filling device, wherein said pressure sensor is able to detect and collect pressure data.

2. The cast equipment according to claim 1, wherein said fixed element comprises a first container and a shape-retaining moldable material inside said first container.

3. The cast equipment according to claim 2, wherein said filling device comprises a second container and an inflatable balloon inside said second container.

4. The cast equipment according to claim 3, wherein said first container and said second container are bags made of weave cloth.

5. The cast equipment according to claim 4, wherein said filling device has a second site attached to said fixed element by weaving said first container and said second container together.

6. The cast equipment according to claim 1, wherein said pressure sensor detects a pressure data through detecting the pressure of said filling device.

7. The cast equipment according to claim 1, wherein said fixed element comprises a separable fasten means and said separable fasten means is a zipper.

8. The cast equipment according to claim 1, wherein said pressure sensor further comprises a component for connecting to an internet environment, and said pressure sensor sends a collected pressure data to a user's device through said internet environment.

9. The cast equipment according to claim 8, wherein said pressure sensor can send an alert to said user's device if said pressure sensor detects a pressure data over a threshold value.

10. The cast equipment according to claim 9, wherein said threshold is 60 mmHg.

11. A method for detecting a pressure data to a casted region, comprising steps of:
(a) applying a fixed element having a separable fasten means to a patient;
(b) fastening a filling device to said fixed element with an attached pressure sensor by said separable fasten means; and
(c) detecting said pressure data by said pressure sensor.

12. The method for detecting a pressure data to a casted region according to claim 11 further comprising a step of sending a detected pressure data to a user's device.

13. The method for detecting a pressure data to a casted region according to claim 12, wherein said detected pressure data is sent through a wireless network environment.

14. The method for detecting a pressure data to a casted region according to claim 11 further comprising a step of sending an alert to a user's device if said pressure data represents a blood blockage.

15. The method for detecting a pressure data to a casted region according to claim 14, wherein said pressure data is a data over 60 mmHg.

16. A method for releasing a blockage to a casted region, comprising steps of:
(a) applying a fixed element having a separable fasten means to a patient;
(b) fastening a filling device to said fixed element with an attached pressure sensor by said separable fasten means;
(c) detecting a blood blockage by said pressure sensor; and
(d) deflating said filling device or removing said fixed element by said separable fasten means to release said blockage.

17. The method for releasing a blockage to a casted region according to claim 16 further comprising a step of sending a detected blockage alert to a user's device after step (c).

18. The method for releasing blockage to a casted region according to claim 17, wherein said detected blockage alert is sent through a wireless network environment.

19. The method for releasing a blockage to a casted region according to claim 16, wherein said filling device is an inflatable balloon.

20. The method for releasing a blockage to a casted region according to claim 16, wherein said blood blockage is a pressure data over 60 mmHg.
